(19) United States
(12) Patent Application Publication
(10) Pub. No.: US 2019/0274645 A1
(43) Pub. Date: Sep. 12, 2019

(54) CANCER BINDING RADIO-OPAQUE PEPTIDES THAT ARE TARGETED FOR DISINTEGRATION BY RADIANT ENERGY

(71) Applicant: CAO GROUP, INC., West Jordan, UT (US)

(72) Inventor: Steven D. Jensen, West Jordan, UT (US)

(73) Assignee: CAO GROUP, INC., West Jordon, UT (US)

(21) Appl. No.: 16/345,066

(22) PCT Filed: Oct. 25, 2017
(86) PCT No.: PCT/US17/58269

§ 371 Date: Apr. 25, 2019

Related U.S. Application Data
(60) Provisional application No. 62/412,945, filed on Oct. 26, 2016.

(51) Int. Cl. A61B 6/00

(52) U.S. Cl. CPC A61B 6/40

(57) ABSTRACT

A compound may comprise a radio-opaque moiety that is readily identifiable by radiography, such as X-rays and the like. The compound may be configured to bind to cancerous cells and minimizes collection within healthy tissue. The compound may readily absorb a wavelength of light that is matched to a radiant energy source that emits light at or near said wavelength.

### CROSS-REFERENCES TO RELATED APPLICATIONS

This application claims the benefit of PCT Application No. PCT/US2017/058269, filed Oct. 25, 2017, which is a non-provisional of U.S. Provisional Application No. 62/412,945 filed on Oct. 26, 2016, which is hereby incorporated by reference in its entirety.

### TECHNICAL FIELD OF THE INVENTION

The present invention discloses cancer binding radio-opaque peptides that are targeted for disintegration by radiant energy and related methods.

### SUMMARY

Embodiments of the present invention provide cancer binding radio-opaque peptides that are targeted for disintegration by radiant energy and related methods.

### DETAILED DESCRIPTION OF THE INVENTION

The discovery of biologically active compounds that target and bind to cancerous cells have provided a novel means to better detect and identify tumorous lesions. Once cancer is identified in a biological host, most often the medical professional will perform surgery to remove it. Surgical removal of a tumorous lesion is risky, because of the risk of dislodging cancerous cells when cutting at or near the tumor, which unfortunately transports them throughout the body via the bloodstream. Dislodged cancer cells most often collect within the lymph nodes where the cancer continues to spread throughout the body. One major function of chemotherapy is to manage the spread of cancer that is caused by dislodged cells after surgery. What are needed are devices and methods that do not require the surgical cutting of the tumor in order to remove it.

When a surgeon removes a cancerous lesion they most often remove a significant portion of healthy tissue surrounding the tumor in order to avoid cutting the tumor itself, as this would in effect dislodge an excessive amount of cancer cells. Contemporary surgical methods intentionally remove large areas of healthy biological tissue when removing cancerous lesions. What are needed are devices and methods that specifically target cancer cells while leaving healthy cells and tissue more or less intact.

The present invention provides a means to radiographically identify and mark cancer cells for destruction, while leaving healthy biological tissue un-marked. The present invention marks cancerous cells so they become more susceptible to disintegration by the absorption of radiant energy than un-marked healthy cells and tissue. The amount of absorbed energy is by design sufficient to destroy the marked cell; the cell becomes in effect burned and exhibits the by-products of combustion. The present invention provides a means to locate and identify/define the tumorous lesion in order to guide the radiant energy source to the treatment site. The present invention provides a means to radiographically locate and identify the tumorous lesion in order to guide the radiant energy source to the treatment site. An embodiment of the present invention comprises the following characteristics all within the same compound:
a) A compound that when introduced into the bloodstream of a host tends to collect and bind to cancerous cells and tissues, while at the same time minimizes the collection within healthy cells and tissues.
b) A compound that readily absorbs a wavelength of light that is matched to a radiant energy source that emits at or near the same wavelength.
c) A radio-opaque moiety that is readily identifiable by radiography such as X-rays and the like.

The present invention utilizes Radio-opaque peptides as biological active compounds that are known to have the ability to collect in tumorous lesions.

The present invention utilizes peptides, polypeptides, polymers, biopolymers and proteins as biological active compounds that are known to have the ability to collect in tumorous lesions. The biological active compounds comprising: peptides, polypeptides, polymers, biopolymers and proteins are generically referred to as “Peptides”. An embodiment of the present invention prefers a group of peptides, polypeptides, polymers, biopolymers and proteins that bind to fibrinogen and fibrin. A list of peptides, polypeptides, polymers, biopolymers and proteins that have an affinity to bind fibrinogen and fibrin are found in U.S. Pat. No. 8,513,380 and is hereby incorporated in its entirety by reference. U.S. Pat. No. 8,513,380 also disclose the means of manufacture and the means to discover additional peptides when applied in practice. When introduced into the blood stream these peptides tend to bind to cancerous cells while leaving healthy cells alone and unbound.

An embodiment of the present invention is designed to flood an organism with Radio-opaque peptides wherein the peptides collect within the cancer cells that in effect mark them for disintegration. Once marked, the cancerous lesion is radiated with radiant energy wherein the bound peptide readily absorbs the incoming radiation and transforms the energy into heat. The cancerous lesion is radiated with sufficient energy such that the peptide-marked cell becomes burned and exhibits the by-products of combustion. An embodiment of the present invention selects a source of radiant energy with a wavelength that is readily absorbed by the peptide wherein the absorption efficiency is 20-100%. Another embodiment of the present invention selects a source of radiant energy with a wavelength that is readily absorbed by the peptide wherein the absorption efficiency is 60-100%.

A preferred embodiment of the present invention selects a radiant energy source that is least likely to be absorbed by healthy biological tissue and at the same time maximizes absorption to the peptide targeting molecule; wherein healthy unmarked cells are less likely to be destroyed by the incoming radiation because they are significantly less absorbent to the radiant energy source; wherein the radiant energy becomes dissipated throughout a deep column of healthy tissue comprising a much larger dissipation area.

By this method a peptide can be introduced into a patient's bloodstream wherein the peptide collects within the cancerous lesion and not within healthy cells. The peptide-marked tumor is then subject to a radiant energy source whose wavelength is selected to maximize the absorption characteristics of the peptide. The cancerous lesion is radiated with sufficient energy such that a portion or all of peptide-marked tumor becomes burned and exhibits the by-products of combustion. The body is then allowed to heal wherein the natural physiological processes of the body remove the destroyed cells. If only a portion of the tumor is radiated, then multiple treatments can be implemented as the tumor is systematically destroyed a portion at a time after a healing interval.

A radio-opaque phospholipid is created by adding a radio-opaque moiety onto a peptide. The present invention utilizes heavy elements as a source of radio-opaque substances such as: iodine, bromine, calcium, barium, strontium, bismuth, tungsten, zirconium, iron, copper, nickel, zinc, silver, tin, gallium, antimony, palladium, rhodium, yttrium, molybdenum, cobalt, chromium, titanium, vanadium, magnesium, gold, platinum, and iridium and any other radiographically visible substance. These radio-opaque substances can be used in their elemental form, as a salt, bound in chelated form, or as an organometallic compound. Any radio-opaque moiety that can be attached to a peptide that does not inhibit the resultant compound's ability to collect and bind to cancer cells is within the scope of this patent. The peptides of the present invention can utilize peptides that are naturally chromatic and/or those peptides that are made chromatic by the addition of a chromatic moiety. A preferred embodiment of the present invention comprises a radio-opaque peptide that is chromatic as well. An embodiment of the present invention has structure:

[(P)(L₍ₘ₎)(R)]ₙ

Wherein P is a peptide, polypeptide, polymer, biopolymer or protein. L is a linkage moiety or polymer such as those listed but not limited to those disclosed under “Crosslinkers” in U.S. Pat. No. 8,513,380. R is a radio-opaque moiety that comprises a radio-opaque heavy element, a radio-opaque organometallic compound, a radio-opaque salt, and/or a radio-opaque metal bound by chelation. M is 0 or 1.N is a number from 1 to 10,000.

Another embodiment of the present invention has structure:

(P)ₙ(L₍ₘ₎)(R)

Wherein P is a peptide, polypeptide, polymer, biopolymer or protein. L is a linkage moiety or polymer such as those listed but not limited to those disclosed under “Crosslinkers” in U.S. Pat. No. 8,513,380.

R is a radio-opaque moiety that comprises a radio-opaque heavy element, a radio-opaque organometallic compound, a radio-opaque salt, and/or a radio-opaque metal bound by chelation. M is a number from 0 to 10,000. N is a number from 1 to 10,000.

The Radio-opaque peptide can be delivered to the organism by way of injection with the appropriate peptide being dissolved in physiological saline or other solution, it can also be delivered orally in tablet or capsule form when blended with the appropriate binding agents, or by any other pharmaceutically accepted method.

The radiant energy source of the present invention comprises both coherent and incoherent sources of radiation. A few embodiments of radiant energy sources include but are not limited to: incoherent light sources such as filament lamps, halogen lamps, fluorescent lamps, plasma lamps and any other incoherent source of light. Coherent sources of light include but are not limited to lasers such as gas lasers, chemical lasers, excimer lasers, solid-state lasers, diode lasers, photonic crystal lasers, dye lasers, fiber lasers, free electron lasers and any other coherent source of light.

The present invention comprises a method that matches the source of radiant energy to the absorption characteristics of a particular radio-opaque peptide compound. First, a radio-opaque peptide compound is selected based upon its absorption characteristics, then a radiant energy source that emits at or near a wavelength that is readily absorbed by the peptide is selected as the preferred source of radiation. An embodiment of the present invention utilizes the absorption lambda max of a peptide as the matching emission wavelength required by the radiant energy source.

The treatment regime would introduce a radio-opaque peptide into the patient's blood stream allowing sufficient time for the peptide to target and bind to the cancerous cells within the tumor. The tumor could then be located and defined by radiography. Based upon analysis of the x-rays, a treatment strategy is planned and executed. Radiant energy from a laser or other radiant energy source would then be focused upon the tumor with sufficient energy such that a portion or all the peptide marked cells become burned and exhibit the by-products of combustion. The body is then allowed to heal wherein the natural physiological processes of the body remove the destroyed cells. If only a portion of the tumor is radiated, then multiple treatments can be implemented as the tumor is systematically destroyed a portion at a time after a healing interval.

The radiant energy can be delivered to the treatment area by direct radiation, a focused beam, a fiber optic cable, or any other means of transmitting radiant energy.

What is claimed is:

1. A compound comprising:
a radio-opaque moiety;
wherein the compound is configured to bind to cancerous cells and minimizes collection within healthy tissue; and
wherein the compound readily absorbs a wavelength of light that is matched to a radiant energy source that emits light at or near said wavelength.
